(19) United States
(12) Patent Application Publication
(10) Pub. No.: US 2026/0306176 A1
(43) Pub. Date: Oct. 8, 2026

(54) SURGICAL SYSTEM WITH CHECKPOINT PLANNING AND NAVIGATION

(71) Applicant: MAKO Surgical Corp., Weston, FL (US)

(72) Inventors: David Reynolds, Rochester, NY (US); Andrew Nelson, New York, NY (US)

(73) Assignee: MAKO Surgical Corp., Weston, FL (US)

(21) Appl. No.: 19/398,779

(22) Filed: Nov. 24, 2025

Related U.S. Application Data
(60) Provisional application No. 63/730,607, filed on Dec. 11, 2024.

(51) Int. Cl. A61B 34/10; A61B 5/00; A61B 34/30

(52) U.S. Cl. CPC A61B 34/10; A61B 5/4509; A61B 34/30; A61B 2034/105; A61B 2034/107

(57) ABSTRACT

A method includes generating, based on patient-specific data, a checkpoint zone of a virtual bone model such that the checkpoint zone corresponds to checkpoint positions compliant with at least one constraint based on the patient-specific data. The method also includes guiding creation or installation of a checkpoint on a physical bone using the checkpoint zone of the virtual bone model, and validating registration of the virtual bone model to the physical bone using the checkpoint.

### CROSS-REFERENCE TO RELATED PATENT APPLICATION

This application claims the benefit of and priority to U.S. Provisional Application No. 63/730,607, filed on Dec. 11, 2024, the entire disclosure of which is hereby incorporated by reference herein.

### BACKGROUND

The present disclosure relates generally to surgical systems for orthopedic surgeries, for example surgical systems that facilitate joint replacement procedures. Joint replacement procedures (arthroplasty procedures) are widely used to treat osteoarthritis and other damage to a patient's joint by replacing portions of the joint with prosthetic components. Joint replacement procedures can include procedures to replace hips, knees, shoulders, or other joints with one or more prosthetic components.

One possible tool for use in an arthroplasty procedure is a robotically-assisted surgical system. A robotically-assisted surgical system typically includes a robotic device that is used to prepare a patient's anatomy to receive an implant, a tracking system configured to monitor the location of the robotic device relative to the patient's anatomy, and a computing system configured to monitor and control the robotic device. Robotically-assisted surgical systems, in various forms, autonomously carry out surgical tasks, provide force feedback to a user manipulating a surgical device to complete surgical tasks, augment surgeon dexterity and precision, and/or provide other navigational cues to facilitate safe and accurate surgical operations.

A surgical plan is typically established prior to performing a surgical procedure with a robotically-assisted surgical system. Based on the surgical plan, the surgical system guides, controls, or limits movements of the surgical device during portions of the surgical procedure. Guidance and/or control of the surgical device serves to assist the surgeon during implementation of the surgical plan. The surgical plan can include multiple stages, for example to be executed on different bones and/or to prepare different surfaces, shapes, recesses, holes, etc. on a given bone, which may typically be executed sequentially. Various features enhancing surgical robot control in scenarios involving multiple surgical stages would be advantageous.

### SUMMARY

One implementation of the present disclosure is a method. The includes generating, based on patient-specific data, a checkpoint zone of a virtual bone model such that the checkpoint zone corresponds to checkpoint positions compliant with at least one constraint based on the patient-specific data. The method also includes guiding creation or installation of a checkpoint on a physical bone using the checkpoint zone of the virtual bone model, and validating registration of the virtual bone model to the physical bone using the checkpoint.

Another embodiment relates to a surgical system. The system includes a robotic device and a circuitry. The circuitry is configured to generate, based on patient-specific data, a checkpoint zone of a virtual bone model such that the checkpoint zone corresponds to checkpoint positions compliant with at least one constraint based on the patient-specific data. The circuitry is also configured to guide creation or installation of a checkpoint on a physical bone using the checkpoint zone of the virtual bone model, and validate registration of the virtual bone model to the physical bone using the checkpoint.

Another embodiment relates to one or more non-transitory computer-readable media storing instructions that, when executed by a processor, cause the processor to perform operations. The operations include generating, based on patient-specific data, a checkpoint zone of a virtual bone model such that the checkpoint zone corresponds to checkpoint positions compliant with at least one constraint based on the patient-specific data. The operations also include guiding creation or installation of a checkpoint on a physical bone using the checkpoint zone of the virtual bone model, and validating registration of the virtual bone model to the physical bone using the checkpoint.

This summary is illustrative only and should not be regarded as limiting.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a perspective view of a femur prepared to receive an implant component, according to an exemplary embodiment.

FIG. 2 is an illustration of a surgical system, according to an exemplary embodiment.

FIG. 3 is a flowchart of a first process that can be executed by the surgical system of FIG. 2, according to an exemplary embodiment.

FIG. 4 is a flowchart of a second process that can be executed by the surgical system of FIG. 2, according to an exemplary embodiment.

FIG. 5 is an illustration of a robotic device, according to an exemplary embodiment.

FIG. 6 is a flowchart of a third process that can be executing by the surgical system of FIG. 2, according to an exemplary embodiment.

FIG. 7 is an illustration of a graphical user interface that can be used with the process of FIG. 6, according to an exemplary embodiment.

### DETAILED DESCRIPTION

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

Presently preferred embodiments of the invention are illustrated in the drawings. An effort has been made to use the same or like reference numbers throughout the drawings to refer to the same or like parts. Although this specification refers primarily to a robotic arm for orthopedic joint replacement, it should be understood that the subject matter described herein is applicable to other types of robotic systems, including those used for non-surgical applications, as well as for procedures directed to other anatomical regions, for example spinal or dental procedures.

### Surgical Robotics System

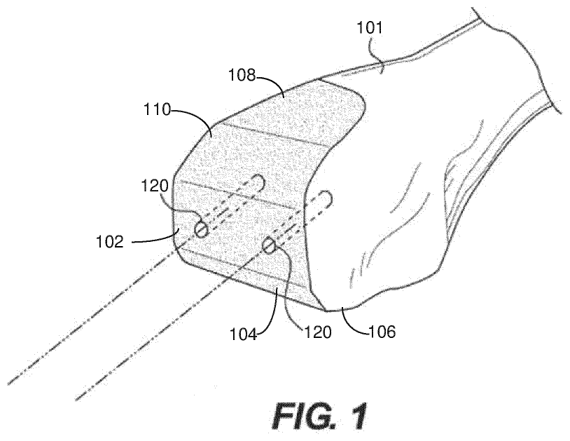

Referring now to FIG. 1, a femur 101 as modified during a knee arthroplasty procedure is shown, according to an exemplary embodiment. As shown in FIG. 1, the femur 101 has been modified with multiple planar cuts. In the example shown, the femur 101 has been modified by five substantially planar cuts to create five substantially planar surfaces, namely distal surface 102, posterior chamfer surface 104, posterior surface 106, anterior surface 108, and anterior chamfer surface 110. The planar surfaces may be achieved using a sagittal saw or other surgical device, for example a surgical device coupled to a robotic device as in the examples described below. The planar surfaces 102-110 are created such that the planar surfaces 102-110 will mate with corresponding surfaces of a femoral implant component. The positions and angular orientations of the planar surfaces 102-110 may determine the alignment and positioning of the implant component. Accordingly, operating a surgical device to create the planar surfaces 102-110 with a high degree of accuracy may improve the outcome of a joint replacement procedure.

As shown in FIG. 1, the femur 101 has also been modified to have a pair of pilot holes 120. The pilot holes 120 extend into the femur 101 and are created such that the pilot holes 120 can receive a screw, a projection extending from a surface of an implant component, or other structure configured to facilitate coupling of an implant component to the femur 101. The pilot holes 120 may be created using a drill, spherical burr, or other surgical device as described herein. The pilot holes 120 may have a pre-planned position, orientation, and depth, which facilitates secure coupling of the implant component to the bone in a desired position and orientation. In some cases, the pilot holes 120 are planned to intersect with higher-density areas of a bone and/or to avoid other implant components and/or sensitive anatomical features. Accordingly, operating a surgical device to create the pilot holes 120 with a high degree of accuracy may improve the outcome of a joint replacement procedure.

A tibia may also be modified during a joint replacement procedure. For example, a planar surface may be created on the tibia at the knee joint to prepare the tibia to mate with a tibial implant component. In some embodiments, one or more pilot holes 120 or other recess (e.g., fin-shaped recess) may also be created in the tibia to facilitate secure coupling of an implant component to the bone.

In some embodiments, the systems and methods described herein provide robotic assistance for creating the planar surfaces 102-110 and the pilot holes 120 at the femur 101, and/or a planar surface and/or pilot holes 120 or other recess on a tibia. It should be understood that the creation of five planar cuts and two cylindrical pilot holes as shown in FIG. 1 is an example only, and that the systems and methods described herein may be adapted to plan and facilitate creation of any number of planar or non-planar cuts, any number of pilot holes, any combination thereof, etc., for preparation of any bone and/or joint in various embodiments. For example, in a hip or shoulder arthroplasty procedure, a spherical burr may be used in accordance with the systems and methods herein to ream a curved surface configured to receive a curved implant cup. Furthermore, in other embodiments, the systems and methods described herein may be used to facilitate placement an implant component relative to a bone (e.g., to facilitate impaction of cup implant in a hip arthroplasty procedure). Many such surgical and non-surgical implementations are within the scope of the present disclosure.

The positions and orientations of the planar surfaces 102-110, pilot holes 120, and any other surfaces or recesses created on bones of the knee joint can affect how well implant components mate to the bone as well as the resulting biomechanics for the patient after completion of the surgery. Tension on soft tissue can also be affected. Accordingly, systems and methods for planning the cuts which create these surfaces, facilitating intra-operative adjustments to the surgical plan, and providing robotic-assistance or other guidance for facilitating accurate creation of the planar surfaces 102-110, other surfaces, pilot holes 120, or other recesses can make surgical procedures easier and more efficient for healthcare providers and improve surgical outcomes.

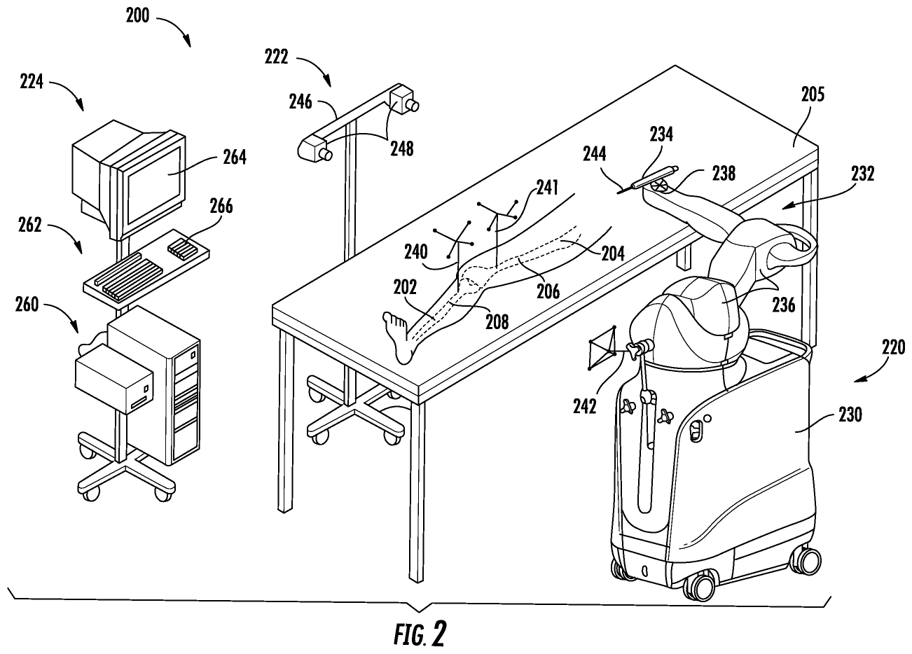

Referring now to FIG. 2, a surgical system 200 for orthopedic surgery is shown, according to an exemplary embodiment. In general, the surgical system 200 is configured to facilitate the planning and execution of a surgical plan, for example to facilitate a joint-related procedure. As shown in FIG. 2, the surgical system 200 is set up to treat a leg 202 of a patient 204 sitting or lying on table 205. In the illustration shown in FIG. 2, the leg 202 includes femur 206 (e.g., femur 101 of FIG. 1) and tibia 208, between which a prosthetic knee implant is to be implanted in a total knee arthroscopy procedure. In other scenarios, the surgical system 200 is set up to treat a hip of a patient, e.g., the femur and the pelvis of the patient. Additionally, in still other scenarios, the surgical system 200 is set up to treat a shoulder of a patient, e.g., to facilitate replacement and/or augmentation of components of a shoulder joint (e.g., to facilitate placement of a humeral component, a glenoid component, and a graft or implant augment). Various other anatomical regions and procedures are also possible.

The robotic device 220 is configured to modify a patient's anatomy (e.g., femur 206 of patient 204) under the control of the computing system 224. One embodiment of the robotic device 220 is a haptic device. “Haptic” refers to a sense of touch, and the field of haptics relates to, among other things, human interactive devices that provide feedback to an operator. Feedback may include tactile sensations such as, for example, vibration. Feedback may also include providing force to a user, such as a positive force or a resistance to movement. One use of haptics is to provide a user of the device with guidance or limits for manipulation of that device. For example, a haptic device may be coupled to a surgical device, which can be manipulated by a surgeon to perform a surgical procedure. The surgeon's manipulation of the surgical device can be guided or limited through the use of haptics to provide feedback to the surgeon during manipulation of the surgical device.

Another embodiment of the robotic device 220 is an autonomous or semi-autonomous robot. “Autonomous” refers to a robotic device's ability to act independently or semi-independently of human control by gathering information about its situation, determining a course of action, and automatically carrying out that course of action. For example, in such an embodiment, the robotic device 220, in communication with the tracking system 222 and the computing system 224, may autonomously complete the series of femoral cuts mentioned above without direct human intervention.

The robotic device 220 includes a base 230, a robotic arm 232, and a surgical tool 234, and is communicably coupled to the computing system 224 and the tracking system 222. The base 230 provides a moveable foundation for the robotic arm 232, allowing the robotic arm 232 and the surgical tool 234 to be repositioned as needed relative to the patient 204 and the table 205. The base 230 may also contain power systems, computing elements, motors, and other electronic or mechanical system necessary for the functions of the robotic arm 232 and the surgical tool 234 described below.

The robotic arm 232 is configured to support the surgical tool 234 and provide a force as instructed by the computing system 224. In some embodiments, the robotic arm 232 allows a user to manipulate the surgical device and provides force feedback to the user. In such an embodiment, the robotic arm 232 includes joints 236 and mount 238 that include motors, actuators, or other mechanisms configured to allow a user to freely translate and rotate the robotic arm 232 and surgical tool 234 through allowable poses while providing force feedback to constrain or prevent some movements of the robotic arm 232 and surgical tool 234 as instructed by computing system 224. As described in detail below according to various embodiments, the robotic arm 232 thereby allows a surgeon to have full control over the surgical tool 234 within a control object while providing force feedback along a boundary of that object (e.g., a vibration, a force preventing or resisting penetration of the boundary). In some embodiments, the robotic arm 232 is configured to move the surgical device to a new pose automatically without direct user manipulation, as instructed by computing system 224, in order to position the robotic arm 232 as needed and/or complete certain surgical tasks, including, for example, cuts in a femur 206.

The surgical tool 234 is configured to cut, burr, grind, drill, partially resect, reshape, and/or otherwise modify a bone. The surgical tool 234 may be any suitable tool, and may be one of multiple tools interchangeably connectable to robotic device 220. For example, as shown in FIG. 2 the surgical tool 234 includes a spherical burr 244. In other examples, the surgical tool 234 may also be a sagittal saw, for example with a blade aligned parallel with a tool axis or perpendicular to the tool axis. The surgical tool 234 may also be a drill, for example with a rotary bit aligned parallel with a tool axis or perpendicular to the tool axis. The surgical tool 234 may also be a holding arm or other support configured to hold an implant component (e.g., cup, implant augment, etc.) in position while the implant component is screwed to a bone, adhered (e.g., cemented) to a bone or other implant component, or otherwise installed in a preferred position. In some embodiments, the surgical tool 234 is an impaction tool configured to provide an impaction force to a cup implant to facilitate fixation of the cup implant to a pelvis in a planned location and orientation.

Tracking system 222 is configured track the patient's anatomy (e.g., femur 206 and tibia 208) and the robotic device 220 (e.g., surgical tool 234 and/or robotic arm 232) to enable control of the surgical tool 234 coupled to the robotic arm 232, to determine a position and orientation of modifications or other results made by the surgical tool 234, and allow a user to visualize the bones (e.g., femur 206, the tibia 208, pelvis, humerus, scapula, etc. as applicable in various procedures), the surgical tool 234, and/or the robotic arm 232 on a display of the computing system 224. The tracking system 222 can also be used to collect biomechanical measurements relating to the patient's anatomy, assess joint gap distances, identify a hip center point, assess native or corrected joint deformities, or otherwise collect information relating to the relative poses of anatomical features. More particularly, the tracking system 222 determines a position and orientation (e.g., pose) of objects (e.g., surgical tool 234, femur 206) with respect to a coordinate frame of reference and tracks (e.g., continuously determines) the pose of the objects during a surgical procedure. According to various embodiments, the tracking system 222 may be any type of navigation system, including a non-mechanical tracking system (e.g., an optical tracking system), a mechanical tracking system (e.g., tracking based on measuring the relative angles of joints 236 of the robotic arm 232), and/or any combination of non-mechanical and mechanical tracking systems.

In the embodiment shown in FIG. 2, the tracking system 222 includes an optical tracking system. Accordingly, tracking system 222 includes a first fiducial tree 240 coupled to the tibia 208, a second fiducial tree 241 coupled to the femur 206, a third fiducial tree 242 coupled to the base 230, one or more fiducials attachable to surgical tool 234, and a detection device 246 configured to detect the three-dimensional position of fiducials (e.g., markers on fiducial trees 240-242). Fiducial trees 240, 241 may be coupled to other bones as suitable for various procedures (e.g., pelvis and femur in a hip arthroplasty procedure). Detection device 246 may be an optical detector such as a camera or infrared sensor. The fiducial trees 240-242 include fiducials, which are markers configured to show up clearly to the optical detector and/or be easily detectable by an image processing system using data from the optical detector, for example by being highly reflective of infrared radiation (e.g., emitted by an element of tracking system 222). In some embodiments, the markers are active light emitting diodes. A stereoscopic arrangement of cameras 248 on detection device 246 allows the position of each fiducial to be determined in 3D-space through a triangulation approach in the example shown. Each fiducial has a geometric relationship to a corresponding object, such that tracking of the fiducials allows for the tracking of the object (e.g., tracking the second fiducial tree 241 allows the tracking system 222 to track the femur 206), and the tracking system 222 may be configured to carry out a registration process to determine or verify this geometric relationship. Unique arrangements of the fiducials in the fiducial trees 240-242 (e.g., the fiducials in the first fiducial tree 240 are arranged in a different geometry than fiducials in the second fiducial tree 241) allows for distinguishing the fiducial trees, and therefore the objects being tracked, from one another.

Using the tracking system 222 of FIG. 2 or some other approach to surgical navigation and tracking, the surgical system 200 can determine the position of the surgical tool 234 relative to a patient's anatomical feature, for example femur 206, as the surgical tool 234 is used to modify the anatomical feature or otherwise facilitate the surgical procedure. Additionally, using the tracking system 222 of FIG. 2 or some other approach to surgical navigation and tracking, the surgical system 200 can determine the relative poses of the tracked bones.

The computing system 224 is configured to create a surgical plan, control the robotic device 220 in accordance with the surgical plan to make one or more bone modifications and/or facilitate implantation of one or more prosthetic components. Accordingly, the computing system 224 is communicably coupled to the tracking system 222 and the robotic device 220 to facilitate electronic communication between the robotic device 220, the tracking system 222, and the computing system 224. Further, the computing system 224 may be connected to a network to receive information related to a patient's medical history or other patient profile information, medical imaging, surgical plans, surgical procedures, and to perform various functions related to performance of surgical procedures, for example by accessing an electronic health records system. Computing system 224 includes processing circuit 260 and input/output device 262. Computing system 224 may include circuitry configured to enable the operations described herein, for example using processing circuit 260 and/or input/output device 262.

The input/output device 262 is configured to receive user input and display output as needed for the functions and processes described herein. As shown in FIG. 2, input/output device 262 includes a display 264 and a keyboard 266. The display 264 is configured to display graphical user interfaces generated by the processing circuit 260 that include, for example, information about surgical plans, medical imaging, settings and other options for surgical system 200, status information relating to the tracking system 222 and the robotic device 220, and tracking visualizations based on data supplied by tracking system 222. The keyboard 266 is configured to receive user input to those graphical user interfaces to control one or more functions of the surgical system 200.

The processing circuit 260 includes a processor and memory device. The processor can be implemented as a general purpose processor, an application specific integrated circuit (ASIC), one or more field programmable gate arrays (FPGAs), a group of processing components, or other suitable electronic processing components. The memory device (e.g., memory, memory unit, storage device, etc.) is one or more devices (e.g., RAM, ROM, Flash memory, hard disk storage, etc.) for storing data and/or computer-readable media for completing or facilitating the various processes and functions described in the present application. The memory device may be or include volatile memory or non-volatile memory. The memory device may include database components, object code components, script components, or any other type of information structure for supporting the various activities and information structures described in the present application. According to an exemplary embodiment, the memory device is communicably connected to the processor via the processing circuit 260 and includes computer-readable media for executing (e.g., by the processing circuit 260 and/or processor) one or more processes described herein, for example non-transitory computer-readable media.

More particularly, processing circuit 260 is configured to facilitate the creation of a preoperative surgical plan prior to the surgical procedure. According to some embodiments, the preoperative surgical plan is developed utilizing a three-dimensional representation of a patient's anatomy, also referred to herein as a “virtual bone model.” A “virtual bone model” may include virtual representations of cartilage or other tissue in addition to bone. To obtain the virtual bone model, the processing circuit 260 receives imaging data of the patient's anatomy on which the surgical procedure is to be performed. The imaging data may be created using any suitable medical imaging technique to image the relevant anatomical feature, including computed tomography (CT), magnetic resonance imaging (MRI), and/or ultrasound. The imaging data is then segmented (e.g., the regions in the imaging corresponding to different anatomical features are distinguished) to obtain the virtual bone model. For example, MRI-based scan data of a joint can be segmented to distinguish bone from surrounding ligaments, cartilage, previously-implanted prosthetic components, and other tissue to obtain a three-dimensional model of the imaged bone.

Alternatively, the virtual bone model may be obtained by selecting a three-dimensional model from a database or library of bone models. In one embodiment, the user may use input/output device 262 to select an appropriate model. In another embodiment, the processing circuit 260 may execute stored instructions to select an appropriate model based on images or other information provided about the patient. The selected bone model(s) from the database can then be deformed based on specific patient characteristics, creating a virtual bone model for use in surgical planning and implementation as described herein.

A preoperative surgical plan can then be created based on the virtual bone model. The surgical plan may be automatically generated by the processing circuit 260, input by a user via input/output device 262, or some combination of the two (e.g., the processing circuit 260 limits some features of user-created plans, generates a plan that a user can modify, etc.). In some embodiments, the surgical plan may be generated and/or modified based on distraction force measurements collected intraoperatively.

The preoperative surgical plan includes the desired cuts, holes, surfaces, burrs, or other modifications to a patient's anatomy to be made using the surgical system 200. For example, for a total knee arthroscopy procedure, the preoperative plan may include the cuts necessary to form, on a femur, a distal surface, a posterior chamfer surface, a posterior surface, an anterior surface, and an anterior chamfer surface in relative orientations and positions suitable to be mated to corresponding surfaces of the prosthetic to be joined to the femur during the surgical procedure, as well as cuts necessary to form, on the tibia, surface(s) suitable to mate to the prosthetic to be joined to the tibia during the surgical procedure. As another example, the preoperative plan may include the modifications necessary to create holes (e.g., pilot holes 120) in a bone. As another example, in a hip arthroplasty procedure, the surgical plan may include the burr necessary to form one or more surfaces on the acetabular region of the pelvis to receive a cup and, in suitable cases, an implant augment. Accordingly, the processing circuit 260 may receive, access, and/or store a model of the prosthetic to facilitate the generation of surgical plans. In some embodiments, the processing circuit facilitate intraoperative modifications to the preoperative plant.

The processing circuit 260 is further configured to generate a control object for the robotic device 220 in accordance with the surgical plan. The control object may take various forms according to the various types of possible robotic devices (e.g., haptic, autonomous). For example, in some embodiments, the control object defines instructions for the robotic device 220 to control the robotic device 220 to move within the control object (e.g., to autonomously make one or more cuts of the surgical plan guided by feedback from the tracking system 222). In some embodiments, the control object includes a visualization of the surgical plan and the robotic device 220 on the display 264 to facilitate surgical navigation and help guide a surgeon to follow the surgical plan (e.g., without active control or force feedback of the robotic device). In embodiments where the robotic device 220 is a haptic device, the control object may be a haptic object as described in the following paragraphs.

In an embodiment where the robotic device 220 is a haptic device, the processing circuit 260 is further configured to generate one or more haptic objects based on the preoperative surgical plan to assist the surgeon during implementation of the surgical plan by enabling constraint of the surgical tool 234 during the surgical procedure. A haptic object may be formed in one, two, or three dimensions. For example, a haptic object can be a line, a plane, or a three-dimensional volume. A haptic object may be curved with curved surfaces and/or have flat surfaces, and can be any shape, for example a funnel shape. Haptic objects can be created to represent a variety of desired outcomes for movement of the surgical tool 234 during the surgical procedure. One or more of the boundaries of a three-dimensional haptic object may represent one or more modifications, such as cuts, to be created on the surface of a bone. A planar haptic object may represent a modification, such as a cut, to be created on the surface of a bone. A curved haptic object may represent a resulting surface of a bone as modified to receive a cup implant and/or implant augment. A line haptic object may correspond to a pilot hole to be made in a bone to prepare the bone to receive a screw or other projection.

In an embodiment where the robotic device 220 is a haptic device, the processing circuit 260 is further configured to generate a virtual tool representation of the surgical tool 234. The virtual tool includes one or more haptic interaction points (HIPs), which represent and are associated with locations on the surgical tool 234. In an embodiment in which the surgical tool 234 is a spherical burr (e.g., as shown in FIG. 2), a HIP may represent the center of the spherical burr. Where one HIP is used to virtually represent a surgical device, the HIP may be referred to herein as a tool center point (TCP). If the surgical tool 234 is an irregular shape, for example as for a sagittal saw, the virtual representation of the sagittal saw may include numerous HIPs. Using multiple HIPs to generate haptic forces (e.g. positive force feedback or resistance to movement) on a surgical device is described in U.S. application Ser. No. 13/339,369, titled “System and Method for Providing Substantially Stable Haptics,” filed Dec. 28, 2011, and hereby incorporated by reference herein in its entirety. In one embodiment of the present invention, a virtual tool representing a sagittal saw includes eleven HIPs. As used herein, references to an “HIP” are deemed to also include references to “one or more HIPs.” As described below, relationships between HIPs and haptic objects enable the surgical system 200 to constrain the surgical tool 234.

Prior to performance of the surgical procedure, the patient's anatomy (e.g., femur 206) is registered to the virtual bone model of the patient's anatomy by any known registration technique. One possible registration technique is point-based registration, as described in U.S. Pat. No. 8,010,180, titled “Haptic Guidance System and Method,” granted Aug. 30, 2011, and hereby incorporated by reference herein in its entirety. Alternatively, registration may be accomplished by 2D/3D registration utilizing a hand-held radiographic imaging device, as described in U.S. application Ser. No. 13/562,163, titled “Radiographic Imaging Device,” filed Jul. 30, 2012, and hereby incorporated by reference herein in its entirety. Registration also includes registration of the surgical tool 234 to a virtual tool representation of the surgical tool 234, so that the surgical system 200 can determine and monitor the pose of the surgical tool 234 relative to the patient (e.g., to femur 206). Registration of allows for accurate navigation, control, and/or force feedback during the surgical procedure.

The processing circuit 260 is configured to monitor the virtual positions of the virtual tool representation, the virtual bone model, and the control object (e.g., virtual haptic objects) corresponding to the real-world positions of the patient's bone (e.g., femur 206), the surgical tool 234, and one or more lines, planes, or three-dimensional spaces defined by forces created by robotic device 220. For example, if the patient's anatomy moves during the surgical procedure as tracked by the tracking system 222, the processing circuit 260 correspondingly moves the virtual bone model. The virtual bone model therefore corresponds to, or is associated with, the patient's actual (i.e. physical) anatomy and the position and orientation of that anatomy in real/physical space. Similarly, any haptic objects, control objects, or other planned automated robotic device motions created during surgical planning that are linked to cuts, modifications, etc. to be made to that anatomy also move in correspondence with the patient's anatomy. In some embodiments, the surgical system 200 includes a clamp or brace to substantially immobilize the femur 206 to minimize the need to track and process motion of the femur 206.

For embodiments where the robotic device 220 is a haptic device, the surgical system 200 is configured to constrain the surgical tool 234 based on relationships between HIPs and haptic objects. That is, when the processing circuit 260 uses data supplied by tracking system 222 to detect that a user is manipulating the surgical tool 234 to bring a HIP in virtual contact with a haptic object, the processing circuit 260 generates a control signal to the robotic arm 232 to provide haptic feedback (e.g., a force, a vibration) to the user to communicate a constraint on the movement of the surgical tool 234. In general, the term “constrain,” as used herein, is used to describe a tendency to restrict movement. However, the form of constraint imposed on surgical tool 234 depends on the form of the relevant haptic object. A haptic object may be formed in any desirable shape or configuration. As noted above, three exemplary embodiments include a line, plane, or three-dimensional volume. In one embodiment, the surgical tool 234 is constrained because a HIP of surgical tool 234 is restricted to movement along a linear haptic object. In another embodiment, the haptic object is a three-dimensional volume and the surgical tool 234 may be constrained by substantially preventing movement of the HIP outside of the volume enclosed by the walls of the three-dimensional haptic object. In another embodiment, the surgical tool 234 is constrained because a planar haptic object substantially prevents movement of the HIP outside of the plane and outside of the boundaries of the planar haptic object. For example, the processing circuit 260 can establish a planar haptic object corresponding to a planned planar distal cut needed to create a distal surface on the femur 206 in order to confine the surgical tool 234 substantially to the plane needed to carry out the planned distal cut.

For embodiments where the robotic device 220 is an autonomous device, the surgical system 200 is configured to autonomously move and operate the surgical tool 234 in accordance with the control object. For example, the control object may define areas relative to the femur 206 for which a cut should be made. In such a case, one or more motors, actuators, and/or other mechanisms of the robotic arm 232 and the surgical tool 234 are controllable to cause the surgical tool 234 to move and operate as necessary within the control object to make a planned cut, for example using tracking data from the tracking system 222 to allow for closed-loop control.

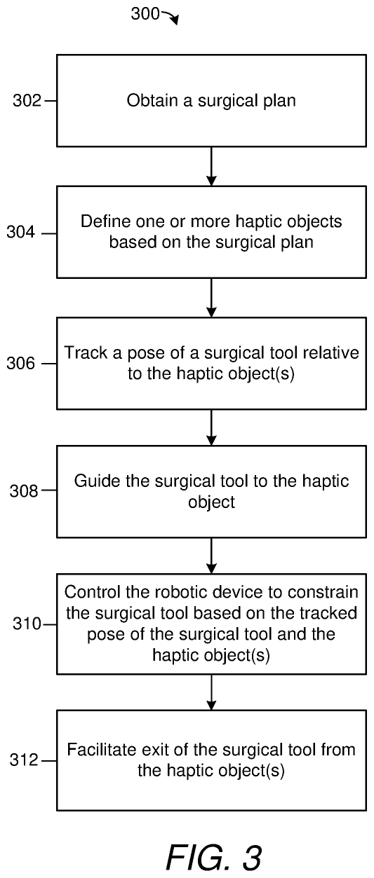

Referring now to FIG. 3, a flowchart of a process 300 that can be executed by the surgical system 200 of FIG. 2 is shown, according to an exemplary embodiment. Process 300 may be adapted to facilitate various surgical procedures, including total and partial joint replacement surgeries.

At step 302, a surgical plan is obtained. The surgical plan (e.g., a computer-readable data file) may define a desired outcome of bone modifications, for example defined based on a desired position of prosthetic components relative to the patient's anatomy. For example, in the case of a knee arthroplasty procedure, the surgical plan may provide planned positions and orientations of the planar surfaces 102-110 and the pilot holes 120 as shown in FIG. 1. The surgical plan may be generated based on medical imaging, 3D modeling, surgeon input, etc.

At step 304, one or more control boundaries, such as haptic objects, are defined based on the surgical plan. The one or more haptic objects may be one-dimensional (e.g., a line haptic), two dimensional (e.g., planar), or three dimensional (e.g., cylindrical, funnel-shaped, curved, etc.). The haptic objects may represent planned bone modifications (e.g., a haptic object for each of the planar surfaces 102-110 and each of the pilot holes 120 shown in FIG. 1), implant components, surgical approach trajectories, etc. defined by the surgical plan. The haptic objects can be oriented and positioned in three-dimensional space relative to a tracked position of a patient's anatomy.

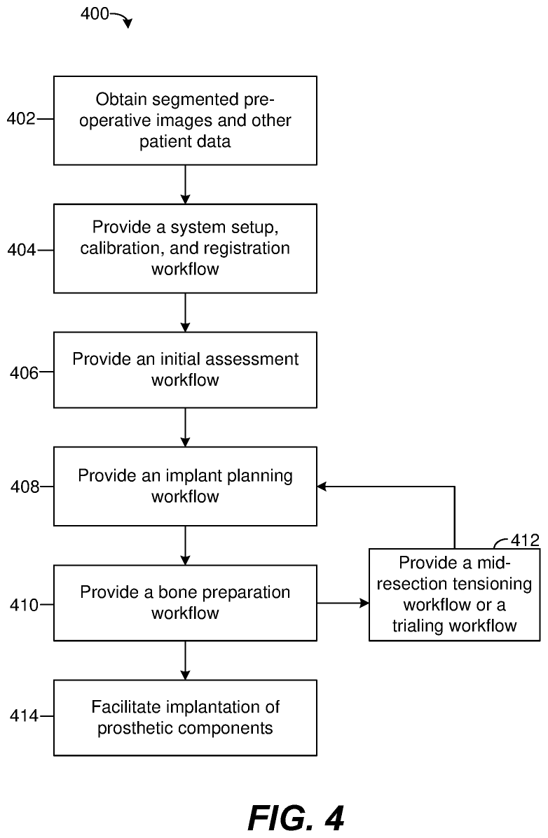

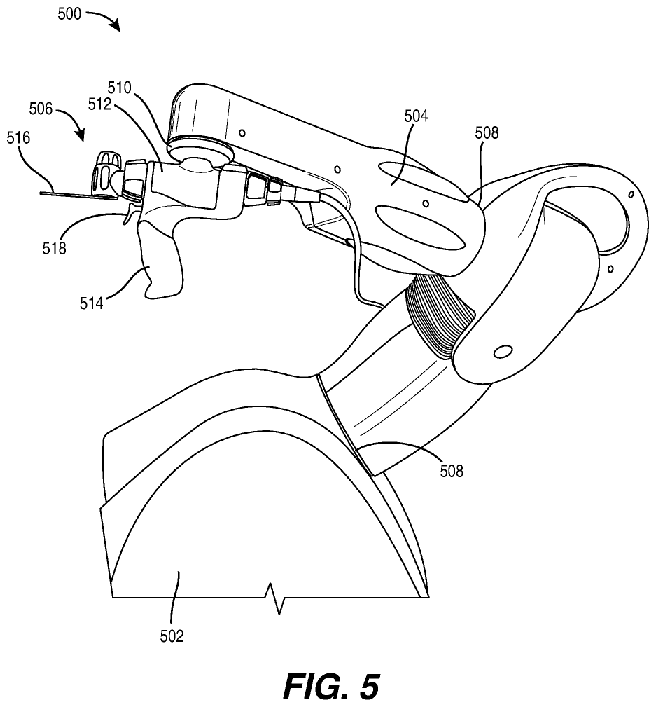

At step 306, a pose of a surgical device is tracked relative to the haptic object(s), for example by the tracking system 222 described above. In some embodiments, one point on the surgical device is tracked. In other embodiments, (e.g., in the example of FIGS. 4-5) two points on the surgical device are tracked, for example a tool center point (TCP) at a tip/effective end of the surgical device and a second interaction point (SIP) positioned along a body or handle portion of the surgical device. In other embodiments, three or more points on the surgical device are tracked. A pose of the surgical device is ascertained relative to a coordinate system in which the one or more haptic objects are defined and, in some embodiments, in which the pose of one or more anatomical features of the patient is also tracked.

At step 308, the surgical device is guided to the haptic object(s). For example, the display 264 of the surgical system 200 may display a graphical user interface instructing a user on how (e.g., which direction) to move the surgical device and/or robotic device to bring the surgical device to a haptic object. As another example, the surgical device may be guided to a haptic object using a collapsing haptic boundary as described in U.S. Pat. No. 9,289,264, the entire disclosure of which is incorporated by reference herein. As another example, the robotic device may be controlled to automatically move the surgical device to a haptic object.

In an embodiment where the robotic device is controlled to automatically move the surgical device to the haptic object (referred to as motorized alignment or automated alignment), the robotic device may be controlled so that a duration of the alignment is bounded by preset upper and lower time thresholds. That is, across various instances of process 300 and multiple procedures, automated alignment in step 308 may be configured to always take between a first amount of time (the lower time threshold) and a second amount of time (the upper time threshold). The lower time threshold may be selected such that the robotic device moves over a long enough duration to be perceived as well-controlled and to minimize collision or other risks associated with high speed. The upper time threshold may be selected such that the robotic device moves over a short enough duration to avoid user impatience and provide improved usability. For example, the upper time threshold hold may be approximately five seconds in an example where the lower time thresholds is approximately three seconds. In other embodiments, a single duration setpoint is used (e.g., four seconds). Step 308 can include optimizing a path for the robotic device such that the step 308 ensures successful alignment to the haptic object while also satisfying the upper and lower time thresholds or duration setpoint.

At step 310, the robotic device is controlled to constrain movement of the surgical device based on the tracked pose of the surgical device and the poses of one or more haptic objects. The constraining of the surgical device may be achieved as described above with reference to FIG. 2.

At step 312, exit of the surgical device from the haptic object(s) is facilitated, e.g., to release the constraints of a haptic object. For example, in some embodiments, the robotic device is controlled to allow the surgical device to exit a haptic object along an axis of the haptic object. In some embodiments, the surgical device may be allowed to exit the haptic object in a pre-determined direction relative to the haptic object. The surgical device may thereby be removed from the surgical field and the haptic object to facilitate subsequent steps of the surgical procedure. Additionally, it should be understood that, in some cases, the process 300 may return to step 308 where the surgical device is guided to the same or different haptic object after exiting a haptic object at step 312.

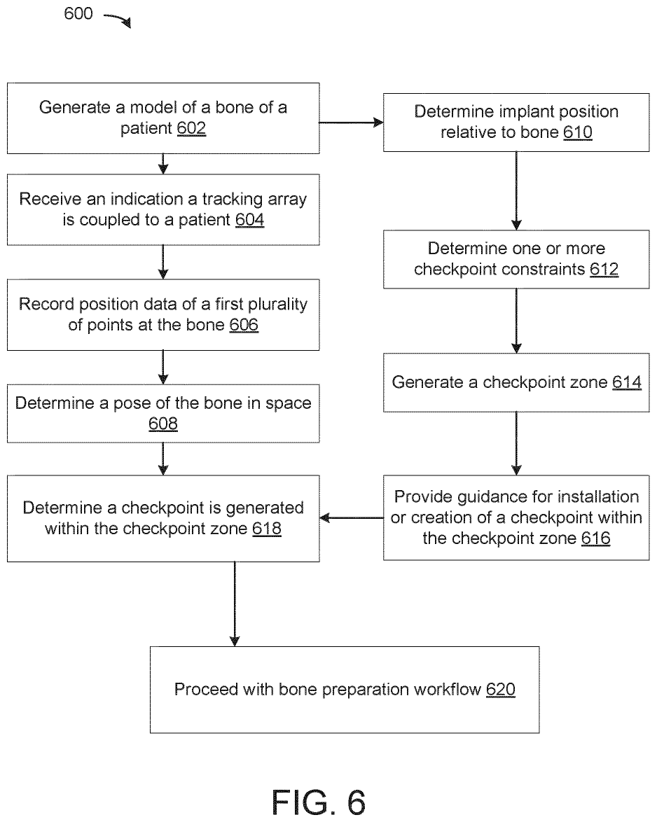

Process 300 may thereby be executed by the surgical system 200 to facilitate a surgical procedure. Features of process 300 are shown in FIGS. 4 and 6 below according to some embodiments, and such features can be combined in various combinations in various embodiments and/or based on settings selected for a particular procedure. Furthermore, it should be understood that the features of FIGS. 4 and 6 may be provided while omitting some or all other steps of process 300. All such possibilities are within the scope of the present disclosure.

Referring now to FIG. 4, a flowchart of a process 400 for facilitating surgical planning and guidance is shown, according to an exemplary embodiment. The process 400 may be executed by the surgical system 200 of FIG. 2, in some embodiments. In some cases, the process 300 is executed as part of executing the process 400.

At step 402, segmented pre-operative images and other patient data are obtained, for example by the surgical system 200. For example, segmented pre-operative CT images or MRI images may be received at the computing system 224 from an external server. In some cases, pre-operative images of a patient's anatomy are collected using an imaging device and segmented by a separate computing system and/or with manual user input to facilitate segmentation. In other embodiments, unsegmented pre-operative images are received at the computing system 224 and the computing system 224 is configured to automatically segment the images. The segmented pre-operative images can show the geometry, shape, size, density, and/or other characteristics of bones of a joint which is to be operated on in a procedure performed using process 400.

Other patient data can also be obtained at step 402. For example, the computing system 224 may receive patient information from an electronic medical records system. As another example, the computing system 224 may accept user input of patient information. The other patient data may include a patient's name, identification number, biographical information (e.g., age, weight, etc.), other health conditions, etc. In some embodiments, the patient data obtained at step 402 includes information specific to the procedure to be performed and the relevant pre-operative diagnosis. For example, the patient data may indicate which joint the procedure will be performed on (e.g., right knee, left knee). The patient data may indicate a diagnosed deformity, for example indicating whether a knee joint was diagnosed as having a varus deformity or a valgus deformity. This or other data that may facilitate the surgical procedure may be obtained at step 402.

At step 404, a system setup, calibration, and registration workflow is provided, for example by the surgical system 200. The system setup, calibration, and registration workflows may be configured to prepare the surgical system 200 for use in facilitating a surgical procedure. For example, at step 404, the computing system 224 may operate to provide graphical user interfaces that include instructions for performing system setup, calibration, and registrations steps. The computing system 224 may also cause the tracking system 222 to collect tracking data and control the robotic device 220 to facilitate system setup, calibration, and/or registration. The computing system 224 may also receiving tracking data from the tracking system 222 and information from the computing system 224 and use the received information and data to calibrate the robotic device 220 and define various geometric relationships between tracked points (e.g., fiducials, markers), other components of the surgical system 200 (e.g., robotic arm 232, surgical tool 234, probe), and virtual representations of anatomical features (e.g., virtual bone models).

The system setup workflow provided at step 404 may include guiding the robotic device 220 to a position relative to a surgical table and the patient which will be suitable for completing an entire surgical procedure without repositioning the robotic device 220. For example, the computing system 224 may generate and provide a graphical user interface configured to provide instructions for moving a portable cart of the robotic device 220 into a preferred position. In some embodiments, the robotic device 220 can be tracked to determine whether the robotic device 220 is properly positioned. Once the cart is positioned, in some embodiments the robotic device 220 is controlled to automatically position the robotic arm 232 in a pose suitable for initiation of calibration and/or registration workflows.

The calibration and registration workflows provided at step 404 may include generating instructions for a user to perform various calibration and registration tasks while operating the tracking system 222 to generate tracking data. The tracking data can then be used to calibrate the tracking system 222 and the robotic device 220 and to register the first fiducial tree 240, second fiducial tree 241, and third fiducial tree 242 relative to the patient's anatomical features, for example by defining geometric relationships between the fiducial trees 240-242 and relevant bones of the patient in the example of FIG. 2. The registration workflow may include tracking a probe used to touch various points on the bones of a joint. In some embodiments, providing the registration workflow may include providing instructions to couple a checkpoint (e.g., a screw or pin configured to be contacted by a probe) to a bone and tracking a probe as the probe contacts the checkpoint and as the probe is used to paint (e.g., move along, touch many points along) one or more surfaces of the bone. The probe can be moved and tracked in order to collect points in or proximate the joint to be operated upon as well as at other points on the bone (e.g., at ankle or hip for a knee surgery).

In some embodiments, providing the registration workflow includes generating instructions to move the patient's leg to facilitate collection of relevant tracking data that can be used to identify the location of a biomechanical feature, for example a hip center point. Providing the registration workflow can include providing audio or visual feedback indicating whether the leg was moved in the proper manner to collect sufficient tracking data. Various methods and approaches for registration and calibration can be used in various embodiments. Step 404 may include steps performed before or after an initial surgical incision is made in the patient's skin to initiate the surgical procedure.

At step 406, an initial assessment workflow is provided, for example by the surgical system 200. The initial assessment workflow provides an initial assessment of the joint to be operated upon based on tracked poses of the bones of the joint. For example, the initial assessment workflow may include tracking relative positions of a tibia and a femur using data from the tracking system while providing real-time visualizations of the tibia and femur via a graphical user interface. The computing system 224 may provide instructions via the graphical user interface to move the tibia and femur to different relative positions (e.g., different degrees of flexion) and to exert different forces on the joint (e.g., a varus or valgus force). In some embodiments, the initial assessment workflow includes determine, by the surgical system 200 and based on data from the tracking system 222, whether the patient's joint has a varus or valgus deformity, and, in some embodiments, determining a magnitude of the deformity. In some embodiments, the initial assessment workflow may include collecting data relating to native ligament tension or native gaps between bones of the joint. In some embodiments, the initial assessment workflow may include displaying instructions to exert a force on the patient's leg to place the joint in a corrected state corresponding to a desired outcome for a joint arthroplasty procedure, and recording the relative poses of the bones and other relevant measurements while the joint is in the corrected state. The initial assessment workflow thereby results in collection of data that may be useful for the surgical system 200 or a surgeon in later steps of process 400.

At step 408, an implant planning workflow is provided, for example by the surgical system 200. The implant planning workflow is configured to facilitate users in planning implant placement relative to the patient's bones and/or planning bone cuts or other modifications for preparing bones to receive implant components. Step 408 may include generating, for example by the computing system 224, three-dimensional computer models of the bones of the joint (e.g., a tibia model and a femur model) based on the segmented medical images received at step 402. Step 408 may also include obtaining three-dimensional computer models of prosthetic components to be implanted at the joint (e.g., a tibial implant model and a femoral implant model). A graphical user interface can be generated showing multiple views of the three-dimensional bone models with the three-dimensional implant models shown in planned positions relative to the three-dimensional bone models. Providing the implant planning workflow can include enabling the user to adjust the position and orientation of the implant models relative to the bone models. Planned cuts for preparing the bones to allow the implants to be implanted at the planned positions can then be automatically based on the positioning of the implant models relative to the bone models.

The graphical user interface can include data and measurements from pre-operative patient data (e.g., from step 402) and from the initial assessment workflow (step 406) and/or related measurements that would result from the planned implant placement. The planned measurements (e.g., planned gaps, planned varus/valgus angles, etc.) can be calculated based in part on data collected via the tracking system 222 in other phases of process 400, for example from initial assessment in step 406 or trialing or tensioning workflows described below with reference to step 412.

The implant planning workflow may also include providing warnings (alerts, notifications) to users when an implant plan violates various criteria. In some cases, the criteria can be predefined, for example related to regulatory or system requirements that are constant for all surgeons and/or for all patients. In other embodiments, the criteria may be related to surgeon preferences, such that the criteria for triggering a warning can be different for different surgeons. In some cases, the computing system 224 can prevent the process 400 from moving out of the implant planning workflow when one or more of certain criteria are not met.

The implant planning workflow provided at step 408 thereby results in planned cuts for preparing a joint to receive prosthetic implant components. In some embodiments, the planned cuts include a planar tibial cut and multiple planar femoral cuts, for example as described above with reference to FIG. 1. The planned cuts can be defined relative to the virtual bone models used in the implant planning workflow at step 408. Based on registration processes from step 404 which define a relationship between tracked fiducial markers and the virtual bone models, the positions and orientations of the planned cuts can also be defined relative to the tracked fiducial markers, (e.g., in a coordinate system used by the tracking system 222). The surgical system 200 is thereby configured to associate the planned cuts output from step 408 with corresponding planes or other geometries in real space.

At step 410, a bone preparation workflow is provided, for example by the surgical system 200. The bone preparation workflow includes guiding execution of one or more cuts or other bone modifications based on the surgical plan created at step 408. For example, as explained in detail above with reference to FIGS. 2-3, the bone preparation workflow may include providing haptic feedback which constrains the surgical tool 234 to a plane associated with a planned cut to facilitate use of the surgical tool 234 to make that planned cut. In other embodiments, the bone preparation workflow can include automatically controlling the robotic device 220 to autonomously make one or more cuts or other bone modifications to carry out the surgical plan created at step 408. In other embodiments, the bone preparation workflow comprises causing the robotic device 220 to hold a cutting guide, drill guide, jig, etc. in a substantially fixed position that allows a separate surgical device to be used to execute the planned cut while being confined by the cutting guide, drill guide, jig, etc. The bone preparation workflow can thus include control of a robotic device in accordance with the surgical plan.

The bone preparation workflow at step 410 can also include displaying graphical user interface elements configured to guide a surgeon in completing one or more planned cuts. For example, the bone preparation workflow can include tracking the position of a surgical device relative to a plane or other geometry associated with a planned cut and relative to the bone to be cut. In this example, the bone preparation workflow can include displaying, in real-time, the relative positions of the surgical device, cut plane or other geometry, and bone model. In some embodiments, visual, audio, or haptic warnings can be provided to indicate completion or start of an event or step of the procedure, entry or exit from a state or virtual object, interruptions to performance of the planned cut, deviation from the planned cut, or violation of other criteria relating to the bone preparation workflow.

In some embodiments, step 410 is provided until all bone cuts planned at step 408 are complete and the bones are ready to be coupled to the implant components. In other embodiments, for example as shown in FIG. 4, a first iteration of step 410 can include performing only a portion of the planned cuts. For example, in a total knee arthroplasty procedure, a first iteration of step 410 can include making a tibial cut to provide a planar surface on the tibia without modifying the femur in the first iteration of step 410.

Following an iteration of the bone preparation workflow at step 410, the process 400 can proceed to step 412. At step 412 a mid-resection tensioning workflow or a trialing workflow is provided, for example by the surgical system 200. The mid-resection tensioning workflow is provided when less than all of the bone resection has been completed. The trialing workflow is provided when all resections have been made and/or bones are otherwise prepared to be temporarily coupled to trial implants. The mid-resection tensioning workflow and the trialing workflow at step 412 provide for collection of intraoperative data relating to relative positions of bones of the joint using the tracking system 222 including performing gap measurements or other tensioning procedures that can facilitate soft tissue balancing and/or adjustments to the surgical plan.

For example, step 412 may include displaying instructions to a user to move the joint through a range of motion, for example from flexion to extension, while the tracking system 222 tracks the bones. In some embodiments, gap distances between bones are determined from data collected by the tracking system 222 as a surgeon places the joint in both flexion and extension. In some embodiments, soft tissue tension or distraction forces are measured. Because one or more bone resections have been made before step 412 and soft tissue has been affected by the procedure, the mechanics of the joint may be different than during the initial assessment workflow of step 402 and relative to when the pre-operative imaging was performed. Accordingly, providing for intra-operative measurements in step 412 can provide information to a surgeon and to the surgical system 200 that was not available pre-operatively and which can be used to help fine tune the surgical plan.

From step 412, the process 400 returns to step 408 to provide the implant planning workflow again, now augmented with data collected during a mid-resection or trialing workflow at step 412. For example, planned gaps between implants can be calculated based on the intraoperative measurements collected at step 414, the planned position of a tibial implant relative to a tibia, and the planned position of a femoral implant relative to a femur. The planned gap values can then be displayed in an implant planning interface during step 408 to allow a surgeon to adjust the planned implant positions based on the calculated gap values. In various embodiments, a second iteration of step 408 to provide the implant planning workflow incorporates various data from step 412 in order to facilitate a surgeon in modifying and fine-tuning the surgical plan intraoperatively.

Steps 408, 410, and 412 can be performed multiple times to provide for intra-operative updates to the surgical plan based on intraoperative measurements collected between bone resections. For example, in some cases, a first iteration of steps 408, 410, and 412 includes planning a tibial cut in step 408, executing the planned tibial cut in step 410, and providing a mid-resection tensioning workflow in step 414. In this example, a second iteration of steps 408, 410, and 412 can include planning femoral cuts using data collected in the mid-resection tensioning workflow in step 408, executing the femoral cuts in step 410, and providing a trialing workflow in step 412. Providing the trialing workflow can include displaying instructions relating to placing trial implants on the prepared bone surfaces, and, in some embodiments, verifying that the trial implants are positioned in planned positions using the tracking system 222. Tracking data can be collected in a trialing workflow in step 412 relating to whether the trial implants are placed in acceptable positions or whether further adjustments to the surgical plan are needed by cycling back to step 408 and making further bone modifications in another iteration of step 410.

In some embodiments, executing process 400 can include providing users with options to jump between steps of the process 400 to enter a desired workflow. For example, a user can be allowed to switch between implant planning and bone preparation on demand. In other embodiments, executing process 400 can include ensuring that a particular sequence of steps of process 400 are followed. In various embodiments, any number of iterations of the various steps can be performed until a surgeon is satisfied that the bones have been properly prepared to receive implant components in clinically-appropriate positions.

As shown in FIG. 4, the process 400 includes step 414 where implantation of prosthetic components is facilitated. Once the bones have been prepared via step 410, the prosthetic components can be implanted. In some embodiments, step 414 is executed by the surgical system 200 by removing the robotic arm 232 from the surgical field and otherwise getting out of the way to allow a surgeon to fix the prosthetic components onto the bones without further assistance from the surgical system 200. In some embodiments, step 414 includes displaying instructions and/or navigational information that supports a surgeon in placing prosthetic components in the planned positions. In yet other embodiments, step 414 includes controlling the robotic arm 232 to place one or more prosthetic components in planned positions (e.g., holding a prosthetic component in the planned position while cement cures, while screws are inserted, constraining an impaction device to planned trajectory). Process 400 can thereby result in prosthetic components being affixed to modified bones according to an intra-operatively updated surgical plan.

Referring now to FIG. 5, a robotic device 500 is shown, according to an exemplary embodiment. In general, the robotic device 500 is configured to modify a patient's anatomy (e.g., femur, tibia, etc.). Robotic device 500 may be an exemplary embodiment of the robotic device 220 as shown in FIG. 2, and may be part of surgical system 200 as shown in FIG. 2. The robotic device 500 includes a base 502, a robotic arm 504, and a surgical device 506. The robotic device 500 may be communicably coupled to a tracking system and a computing system (e.g., tracking system 222 and computing system 224).

The base 502 provides a moveable foundation for robotic arm 504, allowing the robotic arm 504 and the surgical device 506 to be positioned and repositioned as needed relative to a patient. The base 502 may also contain power systems, computing elements, motors, and other electronic or mechanical systems necessary for the functions of the robotic arm 504 and the surgical device 506 described below.

As described above in reference to the robotic device 220 in FIG. 2, the robotic arm 504 is configured to support the surgical device 506 and provide a force as instructed by a computing system (e.g., computing system 224). In some embodiments, the robotic arm 504 allows a user to manipulate the surgical device 506 and provides force feedback to the user. In such an embodiment, the robotic arm 504 includes joints 508 and a mount 510 that includes motors, actuators, or other mechanisms configured to allow a user to freely translate and rotate the robotic arm 504 and surgical device 506 through allowable poses while providing feedback to constrain or prevent some movements of the robotic arm 504 and surgical device 506 as instructed by the computing system 224. In some embodiments, the robotic arm 504 is configured to move the surgical device 506 to a new pose automatically, without direct user manipulation, as instructed by computing system 224 in order to position the robotic arm 504 as desired and/or to complete certain surgical tasks, including modifications to a patient's anatomy (e.g., femur, tibia, etc.).

In some embodiments, the surgical device 506 is configured to cut, burr, grind, drill, partially resect, reshape, and/or otherwise modify a bone. The surgical device 506 may also include a holding arm or other support configured to hold an implant (e.g., acetabular cup, implant augment, etc.), or an impaction tool configured to provide impaction force to a cup implant. The surgical device 506 may also be, or include, any suitable cutting tool (e.g., a drill with a rotary bit, a drill with a spherical burr, a sagittal saw, a sagittal saw blade, a laser cutting device, etc.), and may be, or include, one of multiple tools interchangeably connected to the robotic device 500. For example, as shown in FIG. 5 the surgical device 506 may be a sagittal saw, comprising a housing 512, a handle 514, a sagittal saw blade 516, and a trigger mechanism 518. The housing 512 may be interchangeably connected to mount 510, and may be configured to support the handle 514, sagittal saw blade 516, and trigger mechanism 518. The housing 512 may also contain power systems, computing elements, motors, and other electronic or mechanical systems necessary for the functions of the surgical device 506. The handle 514 may extend from housing 512, and may be configured to allow the user to manipulate the surgical device 506. The handle 514 may be made of any material suitable for cleaning or sterilization. The sagittal saw blade 516 may be interchangeably connected to the housing 512, and may be aligned parallel with the housing 512, or perpendicular to the housing 512 axis. Trigger mechanism 518 may be connected to the housing 512, and can be configured to be pressed (depressed), released, held in place, double-pressed (e.g., pressed, released, and then pressed again in quick succession (e.g., within one second)), or any combination thereof. The trigger mechanism 518 may also be made of any material suitable for cleaning or sterilization, and may interact with the electronic or mechanical systems necessary for the functions of the surgical device 506 located in the housing 512.

### Registration With Checkpoint Planning and Navigation

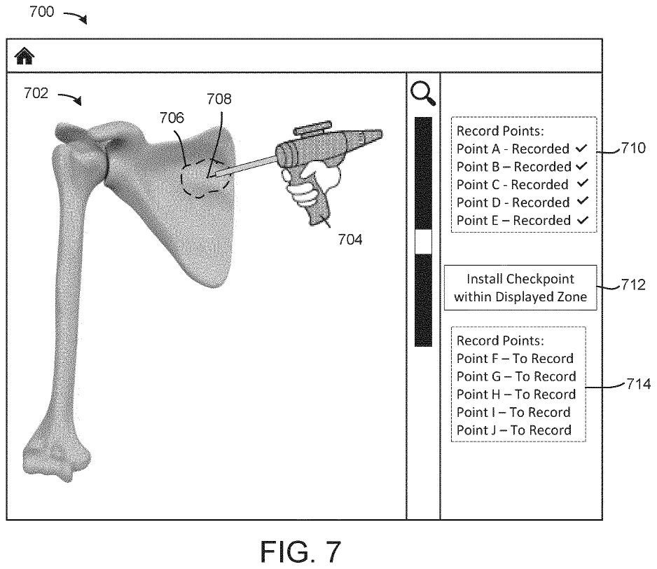

Referring generally to FIGS. 6-7, features are shown which can be provided using the system setup, calibration, and registration workflow at step 404 of process 400 of FIG. 4. In particular, the features of FIGS. 6-7 relate to a portion of step 404 in which one or more checkpoint locations is/are planned and/or registration of one or more structures (e.g., one or more bones, etc.) is performed, for example to allow for tracking of a bone (e.g., using data from a tracking system). As described in detail below, the features of FIGS. 6-7 relate to (i) determining an acceptable area or zone for a checkpoint location and (ii) guiding movement of a surgical tool in generating (e.g., creating, etc.) a checkpoint for a registration or calibration routine (procedure, process, workflow) for the surgical system.

As described herein, a “checkpoint” may refer to a position at a bone, which can be contacted (e.g., via a tracked probe, a surgical device, etc.) to confirm that the checkpoint is positioned where it is expected to be (e.g., relative to a tracking system, a coordinate system, etc.). For example, in some instances where a checkpoint is determined relative to a tracking array, the tracking array may move relative to a bone, which can create a discrepancy (e.g., error, etc.) between a position of the checkpoint relative to (e.g., compared to, etc.) where the checkpoint is expected to be (e.g., relative to the bone, the tracking system, a coordinate system, etc.). By determining and/or generating a checkpoint at a bone, the checkpoint may be fixed relative to the bone, thereby resulting in the checkpoint not moving relative to the bone. Further, the checkpoint may be utilized (e.g., contacted, referenced, etc.) throughout a procedure, for example to validate a registration of a virtual bone model (e.g., to a tracking system, a computing system, etc.), thereby ensuring there is not a discrepancy (e.g., error, difference, etc.) in the tracked setup (e.g., the tracked bone, the tracking system, the coordinate system) from when an initial registration was performed.

Determining an acceptable area or zone of a position of a checkpoint and/or guiding creation of the checkpoint may address various challenges relating to system setup, calibration, and registration (e.g., under step 404). For example, an example challenge associated with determining a position of a checkpoint is that the checkpoint needs to be located and/or positioned where in can be usable (e.g., referenced, accessible, etc.) throughout the procedure. In some instances, a checkpoint may be placed on a portion of a bone that is removed or modified during a procedure, thereby rendering the checkpoint useless. Further, in some instances a checkpoint may be placed at a location that is difficult and/or impractical to access, for example a position that may be obscured by another structure (e.g., where a pin, plate, and/or implant is installed, where a clamp, clasp, and/or retractor is positioned, etc.) throughout the course of a procedure. In certain instances, a checkpoint may be placed at a location where the characteristics of the tissue (e.g., soft, flexible, weak, etc.) where the checkpoint is located results in the checkpoint moving and/or falling out of the surgical field, which renders the checkpoint unusable.

Advantageously, the systems and methods described herein address various challenges relating to determining a position and/or facilitating creation of a checkpoint. For example, the systems and methods described herein define one or more constraints relating to a checkpoint position and/or orientation, for example taking into account one or more considerations (e.g., procedural, anatomical, efficiency, accuracy, reliability, etc.). The one or more constraints can be used to generate a customized (e.g., personalized, tailored, etc.) area, region, or zone, for example associated with an acceptable space a checkpoint can be located (e.g., a space where the checkpoint remains useable throughout the procedure, etc.). Further, the systems and methods described herein can provide guidance (e.g., navigation, feedback, control, etc.) to aid an operator in placing and/or generating the checkpoint. As such, the systems and methods described herein address various problems associated with generic checkpoint determination and/or generation, for example by (i) determining an acceptable area or zone for a checkpoint location and (ii) guiding movement of a surgical tool in generating (e.g., creating, etc.) a checkpoint for a registration or calibration routine (procedure, process, workflow) for the surgical system.

It should be understood that while the systems and methods described herein refer to “a” checkpoint, it is contemplated that in other embodiments the various processes described herein may be implemented using one or more checkpoints, or a plurality of checkpoint positions. Further, it should be understood that while the systems and methods described herein refer to a checkpoint “position,” it is contemplated that in other embodiments the various processes described herein may be implemented using a checkpoint “pose” (e.g., position, orientation, configuration, etc.). In this sense, it is contemplated that in some embodiments the checkpoint is or includes a two-dimensional and/or three-dimensional configuration, for example as a hatch, cross, line, bulb and/or any other suitable 2D or 3D shape.

Referring now to FIG. 6, an example registration workflow or process, shown as process 600, is shown, according to an exemplary embodiment. The process 600 can be executed using the surgical system 200 of FIG. 2, for example by computing system 224 and/or a combination of computing components, robot controllers, processors, etc. as may be included in various robotically-assisted surgical systems (e.g., the robotic device 220, the tracking system 222, the surgical tool 234, etc.). Process 600 can be executed via the robotic device 500 of FIG. 5, for example. Process 600 can be executed as implemented as part of and/or using process 300 and/or process 400, for example in the system setup, calibration, and registration workflow at step 404 of process 400 of FIG. 4.

At step 602, a model is generated. In an exemplary embodiment, the model is a 3-dimensional (3D) model, or virtual model, for example a virtual model of one or more bones of a joint of the patient (e.g., a scapula model, a humerus model, for example of a shoulder joint, etc.). As described herein, the model can be an image-based model generated via segmentation of medical images of a patient's bone, such that the model is a patient-specific virtual model. Further, in some embodiments the model (e.g., virtual bone model, virtual joint model, etc.) is generated (e.g., received, obtained, etc.) via information and/or data associated with the patient.

For example, and as described herein, the surgical system 200 may obtain preoperative data associated with the patient (e.g., images, scans, etc.). The data may include preoperative images obtained via an imaging device (e.g., computed tomography (CT), magnetic resonance imaging (MRI), and/or ultrasound, etc.). The image data may be unsegmented or segmented (e.g., the regions in the imaging corresponding to different anatomical features may be distinguished). For example, scan data of a joint (e.g., CT-based scan data, MRI-based scan data, etc.) can be segmented to distinguish bone from surrounding ligaments, cartilage, previously-implanted prosthetic components, and other tissue. Using the data, the model (e.g., virtual bone model, virtual model of the one or more bones, a virtual model of the joint, etc.) may be generated. In some embodiments, the model is generated and/or obtained via information contained in a database or library. In some embodiments, the model is generated via a selection and/or an input (e.g., provided via a user, an operator, etc. via the input/output device 262) and/or other information provided about the patient.

In some embodiments, the process 600 includes receiving patient data. The patient data may or may not be used in generating the model. As described herein, the patient data may be obtained from a system or device (e.g., a medical records system, etc.), and/or may include, for example, a patient's name, identification number, biographical information (e.g., age, weight, etc.), other health conditions, etc. In some embodiments, the patient data includes information specific to a procedure to be performed and/or the relevant pre-operative diagnosis. For example, the patient data may indicate which joint the procedure will be performed on (e.g., right shoulder, left shoulder, etc.). The patient data may indicate a diagnosed deformity, for example indicating whether a shoulder joint was diagnosed as having a varus deformity or a valgus deformity.

At step 604, an indication is received that a trackable array is coupled to the patient. In some embodiments, the indication that a trackable array is coupled to the patient is received via an input (e.g., a user input provided to the robotic device 220, for example by pressing a button, pushing a foot pedal, input via a graphical interface, etc.). In some embodiments, the tracking system (e.g., the tracking system 222) is used to determine that a trackable array has been coupled to the patient, for example via a pose (e.g., position, location, orientation, etc.) of the trackable array. In other embodiments, the trackable array is provided with a status (e.g., active, etc.), for example indicating that the trackable arrange is or has been coupled with the patient.

In some embodiments, the trackable array (e.g., a fiducial tree, an end effector array, etc.) is coupled to a bone (e.g., a bone of the scapula of the shoulder of the patient, the humerus of the shoulder of the patient, a tibia, a femur, a pelvis, a vertebra, etc.) of a patient. In other embodiments, the trackable array includes a plurality of trackable components, which may be coupled with one or more bones of a joint of the patient (e.g., the scapula and the humerus of a shoulder joint of the patient, etc.). As mentioned above with reference to the example of FIG. 1, a fiducial tree 242 (e.g., tracker base array, etc.) can be coupled with the patient. In some embodiments, the trackable array is incorporated into a probe or a surgical tool (e.g., the surgical tool 234, etc.), for example to track a position of the probe or surgical tool as the probe or surgical tool is held at the one or more bones of the joint. In yet other embodiments, machine vision is used to obtain poses of one or more anatomical structures of the patient (e.g., one or more bones, a bone of a joint, etc.), for example without use of trackable arrays.

At step 606, position data of a plurality of points at the bone is recorded (e.g., collected, determined, etc.). In an exemplary embodiment, a tracking system (e.g., the tracking system 222) is used to record (e.g., collect, determine, etc.) position data of a plurality of points at the bone. For example, the tracking system 222 may be used to track a pose (e.g., position, orientation, etc.) of a probe, for example to record position data of the probe as the probe moves between the plurality of points. Step 606 can include collecting a plurality of points at the bone by recording a position of a tip of the probe as the probe is moved along a surface of the bone (e.g., recording positions in response to clicking of a butting, pushing of a foot pedal, input to a graphical user interface, etc. by a user), so as to record a set points representative of a surface of the bone in real space. In other embodiments, the tracking system 222 is used to track positions of the robotic device 220 (e.g., the surgical tool 234, a tracking probe, a tracking tool, etc.), for example to collect pose data associated with the surgical tool 234 as the surgical tool 234 moves between the plurality of points at the bone.

According to an exemplary embodiment, the position data of the plurality of points are recorded (e.g., collected, received, determined, etc.) and/or ascertained relative to one or more objects and/or components. For example, the position data of the plurality of points may be ascertained (e.g., recorded, collected, determined, etc.) relative to the tracking array and/or an associated coordinate system, as described elsewhere herein. In an exemplary embodiment, the plurality of points at the bone are in one or more different areas (e.g., regions, zones, etc.) at the bone. For example, the plurality of points may include a first set of points (e.g., one, two, three, etc.) within a first area at the bone (e.g., a medial area of the humerus, an anterior area of the scapula, etc.) and a second set of points (e.g., one, two, three, etc.) within a second area at the bone (e.g., a distal area of the humerus, a posterior area of the scapula, etc.). In an exemplary embodiment, the plurality of points includes 10 points; however, in other embodiments the plurality of points includes another suitable number of points (e.g., 5, 7, 8, 15, 20, 25, etc.) and/or is within a suitable range of points (e.g., 5 to 10 points, 10 to 15 points, etc.).

At step 608, a pose of the bone in space is determined, for example by registering the bone model from step 602 to the points collected in step 606 (e.g., by calculating a best fit between the bone model and the points). In an exemplary embodiment, step 608 includes determining the pose (e.g., position, orientation, etc.) of the virtual bone model relative to a tracking array coupled to the bone so as to accurately represent the geometric relationship of the tracking array relative to the physical bone in real/physical space. For example, using the position data of the plurality of points at the bone, pose data associated with the tracking array, and/or the model (e.g., the virtual model of the bone, the joint, etc.), one or more geometric transformations can be defined to enable virtual modeling and tracking which accurately represents the pose of the bone in real/physical space based on tracking of a tracking array coupled to the bone. In this regard, the position data of the plurality of points, the pose data associated with the tracking array, and/or the model may be used to determine a pose of the bone in real/physical space, for example with reference to the virtual coordinate system and/or relative to one or more objects or components (e.g., the tracking array, the tracking system 222, etc.).

Referring still to FIG. 6, at step 610 a position of an implant is determined relative to the bone. For example, and similar to the implant planning workflow of step 408 of FIG. 4, at step 610 a pose (e.g., position, orientation, configuration, etc.) of an implant may be planned relative to the bone, for example in preparation for the bone to receive the implant. It should be understood that step 610 (and steps 612-616 described herein) can occur before, simultaneously with or substantially simultaneously with, and/or after any and/or all of steps 604-608. In some embodiments, step 610 is configured to occur subsequent to step 602 (e.g., generating a model of the bone, etc.). In an exemplary embodiment, using the model of the bone, an implant is selected as part of the implant planning workflow. The implant may be selected (e.g., determined, etc.) based on a surgical plan (e.g., based on one or more anatomical features of a patient, a diagnosis, etc.), as described herein. In an exemplary embodiment, the pose (e.g., pose, orientation, configuration, etc.) of the implant relative to the bone is determined (e.g., planned, etc.) using the bone model and/or the surgical plan.

At step 612, one or more checkpoint constraints is/are determined, for example as spatial limits on the location, orientation, etc. of a checkpoint relative to the bone. Step 612 can include aggregating multiple limitations on positioning of a checkpoint for ease of use of the checkpoint throughout a procedure, for example to account for one or more considerations (e.g., procedural considerations, anatomical considerations, efficiency considerations, accuracy considerations, reliability considerations, etc.). Step 612 can include identifying, as a constraint, a region of bone intended to remain as an exterior surface of the bone after the procedure (i.e., a region not planned for resection and/or for mating against an implant or other component, including, for example, a clamp, clasp, retractor, etc.). Step 612 can include determining, as a checkpoint constraint, a limit relating to an ability to access the checkpoint location throughout the procedure, for example a physical boundary based on a potential of interacting with objects such as other surgical instruments (e.g., tracking arrays and mounting structures thereof, retractors, irrigation tools, cutting tools, jigs, etc.) and/or anatomical features (e.g., other bones, ligaments, muscles, blood vessels, nerves, etc.) with which a checkpoint should avoid interaction. In some embodiments, step 612 can include determining, as one or more checkpoint constraints, one or more regions of a bone which are not physically compatible with receiving a checkpoint, for example based on bone density (e.g., avoiding areas above or below certain densities, etc.), dimensions (e.g., too small or too thin to receive a checkpoint, etc.), load (e.g., based on whether a region of a bone is a high-load-bearing area, etc.), other function of a bone surface (e.g., avoiding articular surfaces of joints, avoiding soft tissue attachment points, etc.), etc., in various embodiments.

In some embodiments, step 612 can additionally or alternative included generating a constraint on a checkpoint position and/or orientation based on an approach area for the surgical procedure (e.g., incision site, incision type, surgical technique, etc.), for example to ensure that an approach area is sufficiently large to allow access to the checkpoint (e.g., a constraint requiring the checkpoint to be in a location accessible via an incision already being made for other purposes relating to the surgical procedure, etc.). In other embodiments, a constraint (e.g., pose, position, location, etc.) on the checkpoint position and/or orientation is determined based on a distance and/or speed associated with movement of one or more surgical tools throughout the procedure (e.g., a probe, the surgical tool 234, etc.), for example to ensure the surgical tool can be moved to the one or more checkpoints in an acceptable amount of time (e.g., less than a threshold duration, etc.) and/or at an acceptable velocity (e.g., less than a threshold velocity, etc.) throughout the procedure. Checkpoint orientation (e.g., an approach axis of checkpoint insertion) can be constrained to avoid skiving, in some embodiments. In some embodiments, the checkpoint zone or orientation is based on the position or orientation of a bony feature, for example the axis of a bone feature such as an acromion is used in some embodiments to constrain the checkpoint orientation (e.g., to within a threshold angle of the orientation of the acromion). In various embodiments, the constraint of the one or more checkpoints is/are determined using any suitable characteristic associated with a suitable consideration (e.g., procedural considerations, anatomical considerations, efficiency considerations, registration reliability considerations, etc.). Any and all combinations thereof (or omission of any set of the above) are within the scope of the present disclosure.

At step 614, a checkpoint zone is generated. According to an exemplary embodiment, the checkpoint zone (e.g., area, space, boundary, etc.) is generated (e.g., determined, produced, obtained, etc.) using the one or more checkpoint constraints, for example to define a physical region which is in compliance with each of the checkpoint constraints identified in step 612 and/or is appropriate for placement of a checkpoint on the bone. For example, a geometric modeling technique, such as constructive solid geometry technique, can be used to determine a three-dimensional region of overlap between acceptable regions indicated by the multiple checkpoint constraints. In this regard, the checkpoint zone generated (e.g., determined, produced, etc.) in step 614 is (e.g., defines, represents, etc.) an acceptable area (e.g., space, boundary, volume, location, etc.) for placement of a checkpoint on the bone without violating (or otherwise by balancing) the one or more checkpoint constraints determined in step 612. In some embodiments, the checkpoint zone is defined by a virtual boundary, for example an outline of an acceptable region on a surface of the bone model, for example such that a region of a surface of the bone model can be highlighted as the region suitable for receiving a checkpoint.

Accordingly, similar to other pose (e.g., position, location, etc.) information described elsewhere herein, the checkpoint zone may be defined in a virtual coordinate system in which the bone model has been registered (e.g., a coordinate system used by the tracking system 222), such that the checkpoint zone determined in step 614 is virtually modelled and can be ascertained in real space using the tracking system 222 together with other components of the surgical system 200 herein (e.g., relative to the tracking array of step 604, and based on registration using the plurality of points at the bone collected in step 606, etc.).

As previously mentioned, the checkpoint zone may define an acceptable region (e.g., area, volume, boundary, etc.) that a checkpoint may be positioned. The checkpoint may provide a position, which establishes a relative position of an anatomical structure associated with the checkpoint (e.g., a bone, a joint, one or more bones, etc.) within the coordinate system (e.g., associated with a surgical field in which the surgical procedure is to be performed). In this regard, the checkpoint may be used not only for an initial registration and/or calibration routine (e.g., to establish or register a pose of the bone relative to a coordinate system, etc.), but also throughout the procedure to determine and/or confirm a pose of the bone relative to the coordinate system (e.g., throughout the procedure).

At step 616, guidance is provided for installation or creation of a checkpoint within the checkpoint zone. As will be described in greater detail below, in some embodiments the tracking system 222 is configured to determine a pose of the robotic device 220 (e.g., the surgical tool 234), for example relative to the bone (e.g., via the coordinate system, the tracking array descried in step 604, etc.). As described herein, the robotic device 220 may provide guidance (e.g., haptic guidance, controlled movement, etc.), for example to guide the surgical tool 234 to and/or within the checkpoint zone. With the surgical tool 234 within the checkpoint zone, a checkpoint may be generated and/or created. As will be described in greater detail herein, the checkpoint may be a hole, divot, scratch, and/or another modification or alteration, for example created in one or more anatomical structures (e.g., the bone, a bone of the joint, an area of the humerus, an area of the scapula, etc.). In other embodiments, the checkpoint is a virtual object, for example such a pose of the surgical tool 234 is captured (e.g., established, set, etc.) at the bone, and a virtual checkpoint is determined relative to the model (e.g., the virtual model, etc.). In some embodiments, if a determination is made that the surgical tool 234 is not within the checkpoint zone, the robotic device 220 may inhibit or prevent one or more operations of the surgical tool 234 (e.g., inhibit or prevent to surgical tool 234 from creating the checkpoint, or divot, etc.), as described elsewhere herein.

In some embodiments, step 616 includes installing a checkpoint as a pin, staple, or other mechanical interface (e.g., as described in U.S. patent application Ser. No. 10/952,796, the entire disclosure of which is incorporated by reference herein) in the checkpoint zone, for example in accordance with virtual guidance presented via a graphical user interface (e.g., as shown in at least FIG. 7). As described herein, guidance can be provided by displaying, for example on the display 264 of the surgical system 200, the virtual model of the bone overlayed with a representation of the checkpoint zone, such that a surgeon can see where on the physical bone the physical checkpoint should be installed for compliance with the checkpoint zone. In some embodiments, a navigation tool (e.g., surgical tool 234, surgical device 506, etc.) for installing the checkpoint as a pin, staple, or other mechanical interface on the bone is tracked by a tracking system 222 in step 616, and displayed on the display 264 relative to the virtual bone model and/or the checkpoint zone in real time to provide navigated guidance facilitating installation of the checkpoint within the checkpoint zone.

In some embodiment, step 616 can include guiding creation of the checkpoint as a divot (depression, hole, notch, etc.) directly in the bone at a location within the checkpoint zone, such that the checkpoint is provided as a modified surface feature of the bone without requiring installation of a separate mechanical device such as a pin on the bone. As described herein, guidance can be provided by displaying, on the display 264 of the surgical system 200, the virtual model of the bone overlayed with a representation of the checkpoint zone, such that a surgeon can see where on the physical bone the checkpoint should be created for compliance with the checkpoint bone. In some embodiments, a navigated tool (e.g., the surgical tool 234, surgical device 506, etc.) for creating the checkpoint is included, for example a sharp-tipped probe including trackable markers such that the navigated tool can tracked by the tracking system 222, and/or its position displayed in real-time relative to the virtual bone model and/or the checkpoint zone, thereby providing visual navigation and guidance that facilitates a surgeon in creating a checkpoint within the checkpoint zone.

In some embodiments, step 616 includes guiding creation of the checkpoint with a drill placing a hole in the bone at a location within the checkpoint zone. In some such embodiments, the checkpoint zone includes bone that will be resected during an initial bone modification step, and the drill is used to create a divot beneath the bone to be resected, i.e., in a bone surface that will be revealed by performance of a bone modification, an inter-cortical divot. In such embodiments, the constraints from step 612 need not prevent the divot from being created in a space overlapping with a planned bone resection, and the divot can be still be used as a checkpoint before and after an overlapping bone resection.

In some embodiments, step 616 includes preparing the bone for checkpoint establishment prior to creating or placing a checkpoint. For example, step 616 can include smoothing or flattening a bone surface to prepare the surface to have a checkpoint established thereon (e.g., a divot created therein) which will be reliably and repeatably found with a probe for later uses a checkpoint. Such preparation can be done robotically, for example by a robot automatically following a cut path for preparing the bone for checkpoint creation and/or by providing haptic feedback to a user operating the robot to prepare the bone. As another example, step 616 can include robotically guiding or automatically providing pre-drilling of a hole to facilitate checkpoint creation or placement (e.g., drilling of a pilot hole to receive checkpoint hardware). These and various other guidance and preparation steps can be provided in any combination in various implementations of step 616.

In some embodiments, in addition to a spatial arrangement of the checkpoint, the checkpoint zone indicates an intended orientation or range of orientations of the checkpoint, for example indicating that the checkpoint should be orthogonal to a bone surface, oriented in an direction aligned with a surgical approach (e.g., facing an incision), aligned to avoid skiving, etc. In such embodiments, step 616 can include providing (e.g., via a display screen, the display 264, etc.) visual guidance for orienting the checkpoint in a desired orientation while placing/creating the checkpoint in the checkpoint zone.

In some embodiments, step 616 includes controlling the robotic device 220 to guide creation or installation of the checkpoint in the checkpoint zone. For example, in some embodiments, the robotic devices provides haptic guidance to guide an end effector of the robotic device 220 (e.g., the surgical tool 234, the surgical device 506, a checkpoint installation tool coupled to the robotic device 220, a checkpoint creation tool coupled to the robotic device 220 etc.) to the bone surface within the checkpoint zone by constraining the end effector from deviating from the checkpoint zone (i.e., providing force feedback forcing the end effector into the checkpoint zone). The haptic guidance may be provided via a virtual boundary established via the checkpoint zone, for example to guide movement of the surgical tool 234 to and/or within the checkpoint zone (e.g., inhibit or limit movement of the surgical tool 234 to outside the checkpoint zone, inhibit or limit operation of the surgical tool unless within the checkpoint zone, etc.). In some embodiments, and as described elsewhere herein, the display 264 of the surgical system 200 may display a graphical user interface instructing a user on how (e.g., which direction, etc.) to move the surgical tool 234 and/or the robotic device 220, for example to bring the surgical tool 234 to within the checkpoint zone (e.g., within the checkpoint zone, etc.). In some embodiments, step 616 includes controlling the robotic device 220 to automatically create or install a checkpoint within the checkpoint zone, for example such that the robotic device 220 provides automated/motorized movement of a checkpoint creation tool to a point in the checkpoint zone and causes the tool to pierce, cut, depress, etc. the bone to create a checkpoint on the bone within the checkpoint zone automatically.

In some embodiments, for example where a tracked tool (e.g., the surgical tool 234, the surgical device 506, etc.) and/or a robot (e.g., the robotic device 220, etc.) is used to create or install the checkpoint, as described in step 616, such data (e.g., tracking data, etc.) can be used to save and/or store the position of the created and/or installed checkpoint on the bone. As will be described below, in other embodiments, or as a validation step (e.g., a verification, confirmation, determination, etc.), the tracked tool (e.g., probe, surgical tool 234, surgical device 506, etc.) can be used to record a position of a point where the checkpoint was created and/or installed, and/or a second plurality of points, for example to validate, save, and/or store the position of the created and/or installed checkpoint on the bone.

At step 618, it is determined that a checkpoint is generated within the checkpoint zone. According to an exemplary embodiment, step 618 is or includes validating and/or verifying the registration performed in step 606 (e.g., registration of the plurality of points relative to the tracking array and/or the coordinate system, etc.). For example step 618 may include recording (e.g., collecting, determining, etc.) position data of a second plurality of points at the bone. According to an exemplary embodiment, the tracking system 222 is used to track a pose (e.g., position, orientation, etc.) of a probe, for example to record position data of the probe as the probe moves to the checkpoint (e.g., a position of the probe at the checkpoint at the bone, etc.). Further, step 618 may include collecting a second plurality of points at the bone by recording a position of a tip of the probe as the probe is moved along a surface of the bone (e.g., recording positions in response to clicking of a butting, pushing of a foot pedal, input to a graphical user interface, etc. by a user), so as to record a second set points representative of a surface of the bone in real space.

According to an exemplary embodiment, step 618 includes comparing the second plurality of points at the bone to the first plurality of points at the bone (e.g., recorded in step 606), for example to validate (e.g., verify, confirm, etc.) the registration of the first plurality of points (e.g., relative to the tracking array, the coordinate system, etc.). In an exemplary embodiment, the comparison of step 618 may validate the registration performed in step 606, for example to validate (e.g., verify, confirm, determine, etc.) that the tracking array has not moved (e.g., relative to the bone, etc.) during the installation and/or creation of the checkpoint (e.g., of step 616). In some embodiments, in response to validating the registration of the plurality of points (e.g., from step 606, etc.), the position of the checkpoint (e.g., as installed or created in step 616, etc.) can be recorded (e.g., saved, stored, collected, determined, etc.), for example for subsequent use throughout the procedure.

According to an exemplary embodiment, at least one of the second plurality of points at the bone is associated with a point at the checkpoint (e.g., installed and/or created at step 616). In some embodiments, the second plurality of points at the bone also include a second set of points at the bone that are in one or more different areas (e.g., regions, zones, etc.) at the bone. In an exemplary embodiment, the second plurality of points are different than the first plurality of points recorded in step 606. For example, the first plurality of points may include 6 points recorded between a medial area of the humerus and a posterior area of the scapula, and the second plurality of points may include 12 points recorded between a distal area of the humerus and an anterior area of the scapula. It should be understood that in other embodiments, the second plurality of points recorded in step 618 may include another suitable number of points (e.g., 5, 7, 8, 15, 20, 25, etc.), may be within a suitable range of points (e.g., 5 to 10 points, 10 to 15 points, etc.), and/or may be within any suitable area or region of the bone, which may be the same and/or different than one or more of he plurality of points recorded in step 606.

In some embodiments, step 618 includes determining that the checkpoint is installed and/or created within the checkpoint zone. For example, step 618 may include comparing the position of the recorded point associated with the checkpoint (e.g., as recorded in step 618) to the checkpoint zone (e.g., area, space, boundary, etc.) generated in step 614. In some embodiments, in response to determining that the checkpoint is positioned at or within a boundary of the checkpoint zone, the system (e.g., the surgical system 200, etc.) may permit the process 600 to proceed. In other embodiments, in response to determining that the checkpoint is positioned outside the checkpoint zone, the system (e.g., the surgical system 200, etc.) may generate a notification (e.g., an alarm, an alert, a warning, etc.) and/or may not permit the process 600 to proceed. For example, a notification may be provided by displaying, for example on the display 264 of the surgical system 200, an instruction (e.g., alert, warning, notification, etc.) to return to step 616 to install and/or create a checkpoint.

At step 620, the process 600 proceeds to a bone preparation workflow. For example, at step 620 the robotic device 220 may be controlled to initiate and/or implement one or more planned cuts (e.g., a humerus cut, a scapula cut, etc.). According to an exemplary embodiment, the robotic device 220 is controlled using the tracked bone position (e.g., via the tracking system 222, etc.) that has been validated using the checkpoint (e.g., validated in step 618). In this regard, and as described herein, the robotic device 220 may be controlled to implement planned cuts that are defined relative to tracked objects (e.g., the tracking array, the coordinate system, etc.) that have been validated via the checkpoint (e.g., in step 618), for example to ensure the planned cuts are associated with corresponding planes and/or geometries in real space.

In some embodiments, following a bone modification (resection, cut, sculpting, etc.) in step 620, process 600 or a portion thereof is executed again to establish an additional checkpoint. For example, in some scenarios, an initial checkpoint is placed in a first iteration of process 600 on a part of a bone that will be resected during a first iteration of step 620, and then process 600 repeats in whole or in part to place an additional (second, subsequent, etc.) checkpoint on the bone, for example on a bone surface revealed by the bone modification performed in the first iteration of process 600. The teachings herein can thus be adapted for moving (e.g., reestablishing) checkpoints over the course of a procedure, for example between cutting steps of an orthopedic surgery.

According to an exemplary embodiment, at one or more times throughout a procedure (e.g., the bone preparation workflow, an implant implantation workflow, etc.), the checkpoint created in process 600 can be used to validate (e.g., verify, confirm, etc.) that a registration is accurate. For example, in response to or following an interruption (e.g., a movement of the bone, a replacement of a tool, etc.), a probe (e.g., the surgical tool 234, the surgical device 506, etc.) may be positioned at the checkpoint. The position of the probe may be recorded and/or compared to a stored and/or saved position associated with the checkpoint (e.g., a position of the checkpoint validated, saved, and/or stored in step 618), for example to validate a relationship between the tracking array and the bone (e.g., validate that the tracking array has not moved relative to the bone, validate a pose of the physical bone relative to the virtual bone model within the tracking system 222 and/or the coordinate system, etc.). In this regard, at one or more times throughout a procedure, the probe (e.g., the surgical tool 234, the surgical device 506, etc.) may be moved to the checkpoint and/or the position of the checkpoint may be recorded and/or validated, for example to validate that the bone registration (e.g., the registration in step 606, the validation in step 618, etc.) is still accurate and/or that the workflow (e.g., bone preparation workflow, implant implantation workflow, etc.) can be initiated (e.g., reinitiated, etc.).

According to an exemplary embodiment, by ensuring that the registration and/or calibration routine (e.g., procedure, process, etc.) is performed from the system-determined/guided (e.g., system-determined, system-generated, etc.) checkpoint, process 600 can reduce or eliminate potential human-caused variations in initiation of the registration and/or calibration routine, which may increase the reliability and/or accuracy of the registration and/or calibration routine. Additionally, by providing guided movement during the checkpoint generation/determination, efficiency and/or usability of the system can be improved. The process 600 thereby provides improvements over alternative approaches to the initial registration and/or calibration routines.

Referring now to FIG. 7, an illustrative graphical user interface, shown as graphical user interface 700, is shown, according to an exemplary embodiment. In an exemplary embodiment, the graphical user interface 700 may be generated and/or displayed (e.g., via the display 264), for example to facilitate the registration workflow or process 600 of FIG. 6. As will be discussed in greater detail, FIG. 7 illustrates a 3-dimensional visualization of a checkpoint zone relative to a virtual bone model, for example to facilitate installation of a checkpoint at a bone.

As shown in FIG. 7, the graphical user interface 700 includes a model, shown as a virtual bone model 702, a virtual checkpoint installation tool, shown as virtual tool 704, and a virtual checkpoint zone, shown as checkpoint zone 706. According to an exemplary embodiment, the virtual bone model 702 is the model that is generated at step 602 of FIG. 6. As described herein, the virtual bone model 702 may be an image-based model generated via segmentation of medical images of a patient's bone, such that the model is a patient-specific virtual model. The virtual tool 704 may be a virtual representation of a checkpoint installation tool (e.g., the surgical tool 234, a drill, pointed probe, etc.), for example coupled with the robotic device 220, etc. According to an exemplary embodiment, the virtual tool 704 illustrates a virtual overlay of a tool relative to the virtual bone model 702, for example such that a surgeon can see where the physical tool (e.g., the surgical tool 234, the drill, etc.) is relative to the physical bone (e.g., via the virtual bone model 702, etc.).

As shown in FIG. 7, the graphical user interface 700 includes a data table, shown as first table 710. In an exemplary embodiment, the first table 710 includes position data of a plurality of recorded points at the bone. For example, the first table 710 may include the position data of the plurality of points at the bone that are recorded (e.g., collected, determined, etc.) at step 606 of FIG. 6. According to an exemplary embodiment, the graphical user interface 700 may illustrate the virtual tool 704 relative to the virtual bone model 702, for example as the physical tool (e.g., the surgical tool 234, etc.) is moved relative to the physical bone to record the positions of the plurality of points (e.g., in real space, etc.). The graphical user interface 700 may include one or more indications (e.g., checkmarks, etc.), for example indicating that the plurality of points at the bone have been recorded.

The graphical user interface 700 is shown to further include the checkpoint zone 706. According to an exemplary embodiment, the checkpoint zone 706 is a virtual representation of the checkpoint zone overlayed on the virtual bone model 702. The checkpoint zone 706 may be the checkpoint zone generated at step 614 of FIG. 6. In this regard, the checkpoint zone 706 may be generated using one or more checkpoint constraints, as described herein. The checkpoint zone 706 may illustrate an acceptable region (e.g., area, volume, boundary, etc.) that a checkpoint may be positioned in. The graphical user interface 700 thereby provides a visualization of a region of the bone in which the checkpoint should be placed, based on the output of steps of process 600.

As shown in FIG. 7, with the checkpoint zone 706 overlayed on the virtual bone model 702, the graphical user interface 700 may include an instruction to install a checkpoint within the displayed zone, shown as instruction 712. For example, following generation of the checkpoint zone, the physical tool (e.g., the surgical tool 234, etc.) may be moved relative to the physical bone, and the graphical user interface 700 may display the virtual tool 704 relative to the virtual bone model 702 and/or the checkpoint zone 706, for example updating the relative positions in real time using data from the tracking system 222. In response to the tool (represented by the virtual tool 704, etc.) being within the checkpoint zone (represented by the checkpoint zone 706) the graphical user interface 700 may provide the instruction 712 to install the checkpoint. As described elsewhere herein, a device (e.g., the robotic device 220, etc.) may provide guidance, for example force feedback to guide the tool to and/or within the checkpoint zone. As shown in FIG. 7, once a checkpoint is installed and/or created, a virtual checkpoint, shown as checkpoint 708, may be shown on the graphical user interface 700. The checkpoint 708 may be provided relative to the virtual bone model 702, the checkpoint zone 706, and/or the virtual tool 704.

As shown in FIG. 7, the graphical user interface 700 includes a data table, shown as second table 714. In an exemplary embodiment, the second table 714 is associated with position data of a second plurality of points at the bone. For example, the second table 714 may be associated with the position data of the second plurality of points at the bone that are recorded (e.g., collected, determined, etc.) at step 618 of FIG. 6. As described herein, the second plurality of points may be used to validate and/or verify a registration of points (e.g., at step 606 of FIG. 6, etc.), validate and/or verify a position of the checkpoint (e.g., at step 614 of FIG. 6, etc.), and/or to store and/or save position data associated with the checkpoint. In some embodiments, the graphical user interface 700 may illustrate the virtual tool 704 relative to the virtual bone model 702, for example as the physical tool (e.g., the surgical tool 234, etc.) is moved relative to the physical bone to record the positions of the second plurality of points (e.g., in real space, etc.). The graphical user interface 700 may include one or more indications (e.g., checkmarks, etc.), for example indicating that the plurality of points at the bone are to be recorded and/or have been recorded.

As described herein, the graphical user interface 700 may include additional, fewer, and/or different components, for example associated with the registration workflow or process 600 of FIG. 6. For example, in some embodiments the graphical user interface 700 illustrates the virtual tool 704 relative to the virtual bone model 702, for example as the physical tool (e.g., the surgical tool 234, etc.) is used to initiate and/or implement one or more steps in a bone preparation workflow (e.g., perform one or more planned cuts, etc.). In this regard, the virtual bone model 702 may be updated, for example to reflect the steps that are initiated and/or implemented as part of the bone preparation workflow (e.g., to reflect the cuts, etc.). Further, throughout the bone preparation workflow the graphical user interface 700 may illustrate the virtual tool 704 relative to the virtual bone model 702 and/or the checkpoint 708, for example when the checkpoint is used to validate (e.g., verify, confirm, etc.) that a registration is still accurate, as described herein. In this regard, the graphical user interface 700 may illustrate the virtual tool 704 relative to the virtual bone model 702 and/or the checkpoint 708 at one or more times throughout a procedure, for example to validate that the bone registration (e.g., the registration in step 606, the validation in step 618, etc.) is still accurate and/or that the workflow (e.g., bone preparation workflow, implant implantation workflow, etc.) can be initiated (e.g., reinitiated, etc.).

### Configuration of Exemplary Embodiments

As utilized herein with respect to numerical ranges, the terms “approximately,” “about,” “substantially,” and similar terms generally mean +/−10% of the disclosed values, unless specified otherwise. As utilized herein with respect to structural features (e.g., to describe shape, size, orientation, direction, relative position, etc.), the terms “approximately,” “about,” “substantially,” and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. For example, the process 600 of the exemplary embodiment described in at least FIG. 6 may be implemented using any components of the surgical system 200 described in at least FIG. 2. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A surgical method, comprising:
generating, based on patient-specific data, a checkpoint zone of a virtual bone model such that the checkpoint zone corresponds to checkpoint positions compliant with at least one constraint based on the patient-specific data;
guiding creation or installation of a checkpoint on a physical bone using the checkpoint zone of the virtual bone model; and
validating registration of the virtual bone model to the physical bone using the checkpoint.

2. The method of claim 1, further comprising performing the registration of the virtual bone model to the physical bone prior to guiding the creation or installation of the checkpoint on the physical bone, wherein guiding the creation or installation of the checkpoint on the physical bone comprises tracking a tool for creating or installing the checkpoint relative to the physical bone and displaying movement of the tool relative to the virtual bone model on a display screen based on the registration.

3. The method of claim 1, further comprising guiding the creation or installation of the checkpoint on the physical bone using the checkpoint zone of the virtual bone model by providing visual feedback facilitating creation of a divot in the physical bone with a tool.

4. The method of claim 1, further comprising:
planning, using the virtual bone model, a pose of an implant relative to the physical bone, and
wherein the at least one constraint associated with the pose of the checkpoint at the physical bone is determined using the planned pose of the implant relative to the physical bone.

5. The method of claim 1, wherein the at least one constraint includes a region of the physical bone that remains as an exterior surface of the physical bone throughout a procedure.

6. The method of claim 1, wherein the at least one constraint includes a region of the physical bone having an anatomical characteristic that is incompatible with the creation or installation of the checkpoint, wherein the anatomical characteristic is a bone density.

7. The method of claim 1, wherein the at least one constraint includes a region associated with potential poses of a surgical instrument throughout a procedure, wherein the potential poses of the surgical instrument are incompatible with a position associated with the creation or installation of the checkpoint.

8. The method of claim 1, further comprising defining a haptic boundary using the checkpoint zone, wherein guiding the creation or installation of the checkpoint comprises operating a robot to provide force feedback inhibiting the creation or installation of the checkpoint outside of the haptic boundary.

9. The method of claim 1, wherein guiding the creation or installation of the checkpoint comprises controlling a robot to automatically create or install the checkpoint in the checkpoint zone.

10. The method of claim 1, wherein validating the registration comprises:
collecting, with a probe at the checkpoint, a first position of the probe relative to a tracking array coupled to the physical bone;
collecting, with the probe at points along a surface of the physical bone, a plurality of second positions of the probe relative to the tracking array coupled to the physical bone; and
storing the first position as the checkpoint position in response to consistency of the plurality of second positions with the registration of the virtual bone model to the physical bone.

11. A surgical system, comprising:
a robotic device; and
circuitry programmed to:
generate, based on patient-specific data, a checkpoint zone of a virtual bone model such that the checkpoint zone corresponds to checkpoint positions compliant with at least one constraint based on the patient-specific data;
guide creation or installation of a checkpoint on a physical bone using the checkpoint zone of the virtual bone model; and
validate registration of the virtual bone model to the physical bone using the checkpoint.

12. The surgical system of claim 11, wherein the circuitry is further programmed to:
perform the registration of the virtual bone model to the physical bone prior to guiding the creation or installation of the checkpoint on the physical bone, wherein guiding the creation or installation of the checkpoint on the physical bone comprises tracking a tool for creating or installing the checkpoint relative to the physical bone and displaying movement of the tool relative to the virtual bone model on a display screen based on the registration.

13. The surgical system of claim 11, wherein the circuitry is further programmed to:
plan, using the virtual bone model, a pose of an implant relative to the physical bone, and
wherein the at least one constraint associated with the pose of the checkpoint at the physical bone is determined using the planned pose of the implant relative to the physical bone.

14. The surgical system of claim 11, wherein the at least one constraint includes a region of the physical bone that remains as an exterior surface of the physical bone throughout a procedure.

15. The surgical system of claim 11, wherein the at least one constraint includes a region of the physical bone having an anatomical characteristic that is incompatible with the creation or installation of the checkpoint, wherein the anatomical characteristic is a bone density

16. The surgical system of claim 11, wherein the at least one constraint includes a region associated with potential poses of a surgical instrument throughout a procedure, wherein the potential poses of the surgical instrument are incompatible with a position associated with the creation or installation of the checkpoint.

17. The surgical system of claim 11, wherein the circuitry is further programmed to:
define a haptic boundary using the checkpoint zone, wherein guiding the creation or installation of the checkpoint comprises operating the robotic device to provide force feedback inhibiting the creation or installation of the checkpoint outside of the haptic boundary.

18. The surgical system of claim 11, wherein guiding the creation or installation of the checkpoint comprises controlling the robotic device to automatically create or install the checkpoint in the checkpoint zone.

19. One or more non-transitory computer-readable media storing instructions that, when executed by a processor, cause the processor to perform operations comprising:
generating, based on patient-specific data, a checkpoint zone of a virtual bone model such that the checkpoint zone corresponds to checkpoint positions compliant with at least one constraint based on the patient-specific data;
guiding creation or installation of a checkpoint on a physical bone using the checkpoint zone of the virtual bone model; and
validating registration of the virtual bone model to the physical bone using the checkpoint.

20. The one or more non-transitory computer-readable media of claim 19, wherein the instructions, when executed by the processor, cause the processor to perform operations comprising:
performing the registration of the virtual bone model to the physical bone prior to guiding the creation or installation of the checkpoint on the physical bone, wherein guiding the creation or installation of the checkpoint on the physical bone comprises tracking a tool for creating or installing the checkpoint relative to the physical bone and displaying movement of the tool relative to the virtual bone model on a display screen based on the registration.
